(19) United States
(12) Patent Application Publication
(10) Pub. No.: US 2018/0256402 A1
(43) Pub. Date: Sep. 13, 2018

(54) SCAFFOLD-BASED WOUND CARE DELIVERY SYSTEM AND METHOD

(71) Applicant: Hollister Incorporated, Libertyville, IL (US)

(72) Inventor: ABRAM D. JANIS, LIBERTYVILLE, IL (US)

(73) Assignee: Hollister Incorporated, Libertyville, IL (US)

(21) Appl. No.: 15/759,303

(22) PCT Filed: Sep. 19, 2016
(86) PCT No.: PCT/US2016/052480

§ 371 Date: Mar. 12, 2018

Related U.S. Application Data
(60) Provisional application No. 62/219,946, filed on Sep. 17, 2015.

(51) Int. Cl. A61F 13/00; A61L 27/36; A61L 27/54; A61M 35/00

(52) U.S. Cl. CPC A61F 13/00012; A61F 13/00029; A61F 13/00063; A61F 13/00068; A61L 27/3633; A61L 27/54; A61M 2205/3303; A61L 2300/404; A61L 2300/426; A61M 2205/7545; A61M 2202/07; A61M 2205/3368; A61M 2205/3324; A61M 35/00

(57) ABSTRACT

A bioactive scaffold for cell delivery to a wound includes a degradable body portion configured to be placed within a wound bed, the body portion including a least one extracellular matrix biomaterial.

### CROSS REFERENCE TO RELATED APPLICATIONS

This application is the national stage of International Application No. PCT/US2016/052480 filed Sep. 19, 2016, which claims priority to U.S. Provisional Application Ser. No. 62/219,946, filed on Sep. 17, 2015, the entire contents of which are incorporated herein by reference.

### FIELD OF THE INVENTION

The present invention relates generally to wound care, and more particularly to bioactive scaffold-based delivery systems and methods for dermal and internal wounds.

### BACKGROUND OF THE INVENTION

Chronic wounds are a large and increasingly important public health crisis. These wounds are often the symptomatic manifestations of underlying disease, which can include but are not limited to diabetes or diabetic neuropathy, vascular valvular dysfunction, or tissue damage or necrosis from pressure or shear. Acute traumatic, avulsive injury and first, second and third burns also lead to a large proportion of wounds. In response, many dressings and therapies have been proposed, some of which utilize synthetic or naturally derived scaffolds and allogeneic or autologous cells or tissues, or a combination of the scaffolds and cells or tissues.

While scaffolds are relatively stable, even in the inhospitable chronic or traumatic wound microenvironment, the same is not true for cellular therapies. It is well known that healthy mammalian cells depend on extracellular matrix (“ECM”) attachment for survival/performance and can be negatively affected by the cytokines and ECM breakdown products of the inflammatory milieu. The delivery of allogeneic or autologous cells to a chronic wound or a diseased implant site is thought to be equivalent to planting “good seeds in bad soil.” The same holds true for large traumatic wounds and burns, in particular third degree burns. Indeed, in many cell-based wound treatment systems the delivery means and the acellular materials to be delivered are developed independently of one another.

In view of the above, there is a need for scaffold delivery systems that have an enhanced degree of cytocompatibility between the scaffold and the materials being delivered to a wound cite to increase cellular survival, proliferation, engraftment and general system efficacy.

### SUMMARY OF THE INVENTION

In an embodiment, a bioactive scaffold for cell delivery to a wound includes a degradable body portion configured to be placed within a wound bed, the body portion including a least one extracellular matrix biomaterial.

In another embodiment, a bioactive scaffold for cell delivery to a wound includes a degradable, flexible and porous tubular body portion configured to be placed within a wound bed, the body portion including an extracellular matrix biomaterial, wherein the tubular body portion has a substantially hollow interior.

In yet another embodiment, a method of delivering cells to a wound to facilitate healing includes placing a bioactive scaffold for cell delivery within a wound bed, the scaffold having a degradable body portion that includes at least one extracellular matrix biomaterial and delivering cells to the wound via the body portion of the scaffold.

### BRIEF DESCRIPTION OF THE DRAWINGS

The present invention will be better understood from reading the following description of non-limiting embodiments, with reference to the attached drawings, wherein below:

FIG. 1 is a perspective view of a degradable scaffold for the delivery of cells according to an embodiment of the invention;

FIG. 2 is a perspective view of a cell suspension being added to the degradable scaffold of FIG. 1;

FIGS. 3A and 3B are perspective views of single or multi layered scaffolds, respectively, with voids and inlet and/or outlet ports for the addition and delivery of cells, according to embodiments of the invention.

FIGS. 4A and 4B are perspective views of a scaffold with a passage and inlet and/or outlet ports, and the movement of cells therethrough, and a filter, respectively, according to an embodiment of the invention.

FIGS. 5A-5D are various perspective and planar views of a tubular scaffold for the delivery of cells according to another embodiment of the invention.

FIGS. 6A-6C are various perspective and planar views of a method of utilizing the tubular scaffold of FIGS. 5A-5D.

FIG. 7 is a planar view of a scaffold having a tubular body portion that has been formed into a coil according to yet another embodiment of the invention.

FIGS. 8A-8C are planar and perspective views of a method of use of the tubular scaffold of FIG. 7.

FIG. 9 is a perspective view of a multi-layer cell delivery scaffold according to an embodiment of the invention.

FIG. 10 is a perspective view of a multi-layer cell delivery scaffold according to another embodiment of the invention.

FIGS. 11A and 11B are perspective and enlarged views of the multi-layer scaffold of FIG. 10.

FIGS. 12A-12C are various perspective views of a cell delivery scaffold utilized with a cover dressing according to an embodiment of the invention.

### DETAILED DESCRIPTION

Reference will be made below in detail to exemplary embodiments of the invention, examples of which are illustrated in the accompanying drawings. Wherever possible, the same reference characters used throughout the drawings refer to the same or like parts, without duplicative description.

As used herein, the terms “substantially,” “generally,” and “about” indicate conditions within reasonably achievable manufacturing and assembly tolerances, relative to ideal desired conditions suitable for achieving the functional purpose of a component or assembly. Additionally, as will be appreciated, embodiments of the present invention may be used to treat animal tissue, and other materials generally, and are not limited to human tissue.

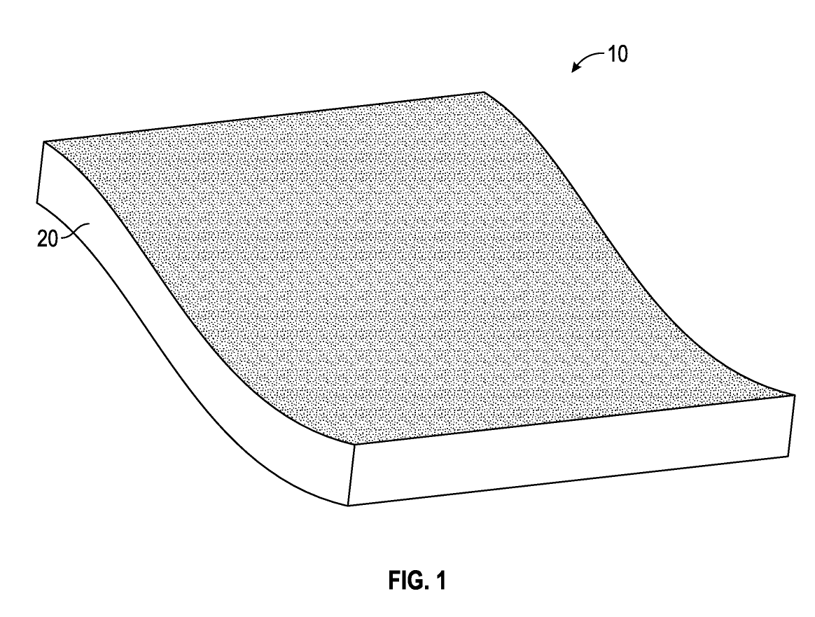

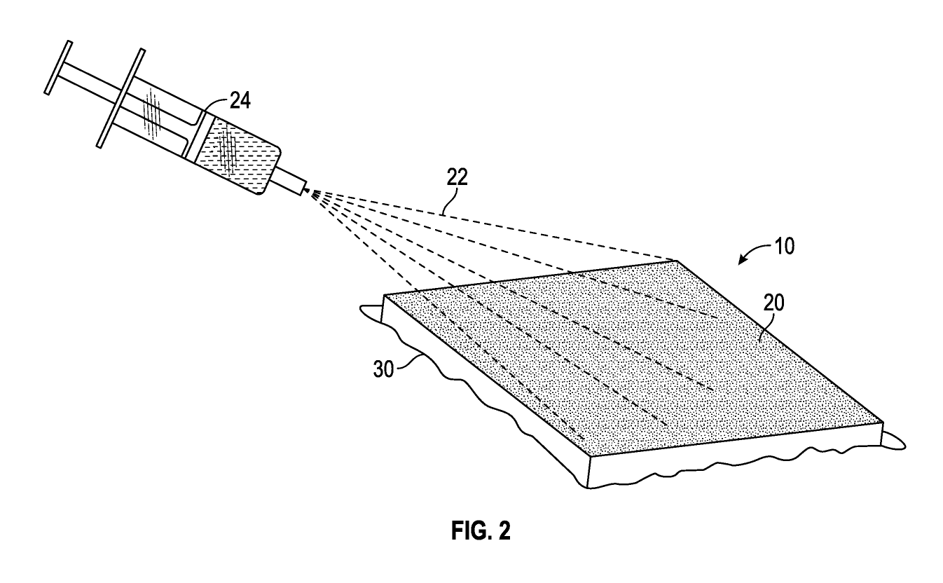

Referring generally to FIGS. 1 and 2, a degradable bioactive scaffold 10 includes a body portion 20 that is configured to receive, contain, and deliver cells 22 to an injury site 30, such as a wound or the like. The degradable scaffold 10 includes, or is composed of, extracellular matrix biomaterials, i.e., the materials produced intracellularly by resident cells and secreted into the ECM. In embodiments, the ECM biomaterials include proteins, carbohydrates, proteoglycans, glycosaminoglycans, synthetic materials and/or synthetic materials functionalized with attachment sequence peptides and/or cytokines (growth factors), which improve cell survival and engraftment and stimulate cell proliferation. As such, when the scaffold 10 degrades and/or elutes cytokines, it improves the recipient injury or wound site; in this analogy, the degrading scaffold 10 serves as “fertilizer”, improving the “soil” of the wound 30 for the administration of the cells 22, i.e., “seeds.”

While shown in a rectangular configuration, it will be appreciated that other shapes and sizes of the body portion may be employed depending on wound characteristics. The body portion may be a porous structure with micro or macro pores.

In specific embodiments, the degradable scaffold 10 may include, alone or in combination, xenogeneic extracellular matrix biomaterials with preserved or altered structures and preserved or augmented cytokines (growth factors). Examples of xenogeneic mammalian or allogeneic or autologous human ECM biomaterials include, but are not limited to, collagen, elastin, fibronectin, laminin, fibrin, hyaluronic acid, invertebrate chitosan, or reconstituted or decellularized stomach submucosa, small intestinal submucosa, urinary bladder matrix, urinary bladder submucosa, forestomach submucosa, dermis, spleen ECM, lung ECM, gall bladder ECM, pancreatic ECM, or serous or visceral pericardium.

The ECM biomaterials may also include or be composed of purified and decellularized membranous or reconstituted collagen(s) from vertebrate and invertebrate sources, including but not limited to human, porcine, bovine, ovine, piscine, marsupial, or Cnidarian (jellyfish) sources. Recombinant human proteins expressed in bacteria or plants, then electrospun, cast, or 3D printed may be included. Bacterial cellulose or alginate may be present or a plant derived extracellular matrix, including, but not limited to, electrospun soy protein, soybean paste, soybean hydrogel, soy-protein, agarose, alginate, cellulose hydrogels, electrospun cellulose as well as bacterial or invertebrate chitin, which may be further processed into chitosan. Human allografts (cadaveric tissue) or amnion, chorion or umbilical tissue; fibrin glue may also be employed. Human autografts (partial thickness skin grafts or recovered surgically removed tissue), for example platelet rich plasma, fibrin, may also be used. Cultured human tissue (allogeneic or autologous), hyaluronic acid (cast or electrospun) or other glycosaminoglycans, cast or electrospun, proteoglycans (ex. aggrecan, chondroitin sulfate), cast or electrospun, may also be utilized. Embodiments may include the incorporation of synthetic degradable polymers to modulate performance.

Referring specifically to FIG. 2, as mentioned, the scaffold 10 is configured to deliver cells 22 to a wound 30. In the depicted embodiment, the cells 22 may be contained within a delivery fluid and may be applied to the body portion 20 via an aerosolized spray or like external delivery means 24. As discussed in greater detail below, other delivery means can be utilized with various embodiments.

The cells to be delivered may be selected to address a specific etiology underlying the pathology of a wound. In embodiments, the cell source may include allogeneic or autologous cells, including, but not limited to, autologous adipose derived stem cells, liver parenchyma, stromal vascular fraction, keratinocytes, dermal fibroblasts, platelet rich plasma, circulating stems cells, bone marrow aspirate, or other tissue specific stem cells and allogeneic cells including but not limited to fibroblasts, epidermal keratinocytes and mixed cell populations such as those included in adipose, cord blood or bone marrow allografts or autografts. Non-immunogenic xenografts from non-human mammalian species could also be delivered in the system. In the case of an internally deployed scaffold, cells could include for example, stomach or intestinal epithelium, stroma, pancreatic islet cells, smooth or skeletal muscle. The cell source may include cultured allogeneic cells including but not limited to dermal fibroblasts, keratinocytes, muscle, and digestive epithelium.

Moreover, in certain embodiments, the scaffold 10 can also be used to deliver growth medium including but not limited to serum or platelet rich plasma from the recipient or medium supplemented with autologous serum or platelet rich plasma. Flowable ECM biomaterials such as hyaluronic acid or ECM gels could also be used to supply cells to the scaffold 10 for subsequent delivery to a wound bed.

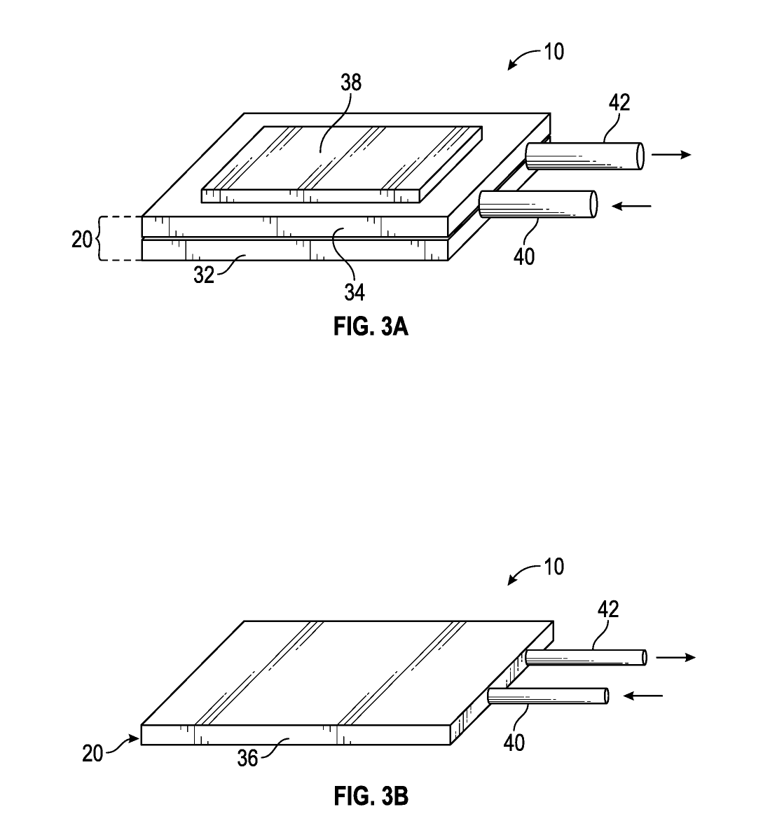

Referring now to FIGS. 3A and 3B, in certain embodiments, the body portion 20 can include multiple layers, e.g., first and second layers, 32 and 34 respectively, or a single layer 36 (FIG. 3B) modified to enclose cells. In aspects, the layers 32 and 34 of the scaffold 10, or a single layer 36, can be glued, ultrasonically welded, laser welded, thermally melted, in a manner that does not ablate bioactivity, or cross-linked to create voids for containment of cells or cell suspensions. In certain embodiments, one or more layers of the body portion 20 may be formed or constructed with a void or compartment 38 configured to contain the cells to be delivered. The layers may be constructed from the same ECM biomaterials or biomaterials having different properties, e.g., porosity, functionality, etc. In embodiments, one of the layers may be non-degradable.

Crosslinking can be achieved through the addition of transglutaminase, 1-Ethyl-3-(3-dimethyl aminopropyl)-carbodiimide, genipin, tannic acid, nordihydroguaiaretic acid, N-hydroxy-succinimide, D-ribose, octadiazopyruvoyl polyamidoamine dendrimer, glucose or other reducing sugars (with or without ultraviolet (UV) light), glutaraldhyde, or photoactive dyes including but not limited to as indocyanine green, methylene blue or rose bengal and respective correlated specific wavelength ranges of light energy, or just UV light alone.

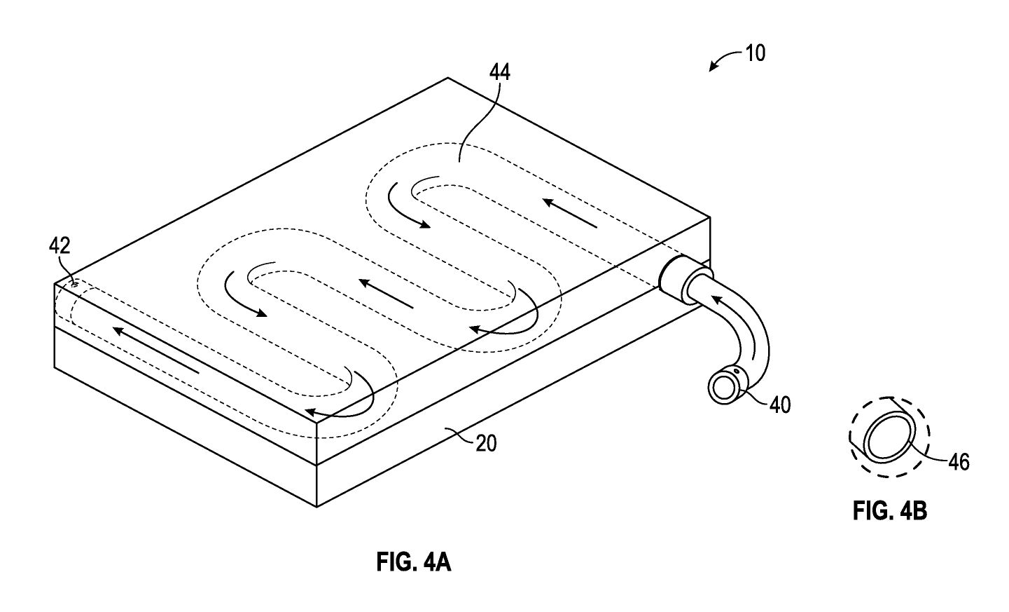

Continuing to refer to FIGS. 3A and 3B, the bioactive scaffold 10 may further include an inlet 40 and an outlet 42 port. The ports may be operatively connected to the void or compartment 38 to deliver cells to the same. An optional filter (FIG. 4B) may be utilized on the outlet port to retain cells within the scaffold 10 while allowing passage of the liquid cell delivery medium through the scaffold prior to placement in a wound. In embodiments, the cells may be delivered to and contained within a porous structure of the layers 32, 34, 36 for delivery to the wound site. As will be appreciated, in such embodiments, the cells would be delivered, e.g., injected, into the inlet via suitable delivery means such as a syringe.

Turning to FIGS. 4A and 4B, in embodiments, the scaffold 10 can include an inlet 40 and an outlet 42 that are operatively connected to a passage 44. The passage 44 may be a porous structure that allows cells to pass into the body portion 20 of the scaffold for delivery. The outlet 42 may include a filter 46, which permits passage of cell delivery media or vehicle, while preventing the release of cells. The filter 46 may also be modified, e.g., partially obstructed, to offer resistance to flow in order to increase the internal hydrostatic pressure within the tube to force cell suspension into the potentially microporous walls of the passage 44.

The passage 44 through the scaffold 10 can be constructed from the scaffold itself, i.e., integral to the scaffold and made from the same ECM biomaterial, or could be a separate construct composed of another degradable, preferably bioactive material, e.g., a second, different ECM biomaterial. The secondary material may persist in the final scaffold/device, or may be sacrificial to create voids in the primary material. In embodiments, the bioactive scaffold can be fabricated in dried or hydrated form and deployed hydrated or rehydrated in situ. The passage 44 is porous to the extent that cells may pass through into the body portion 20 of the scaffold 10.

In certain embodiments, the body portion 20 may have a layer or a cover to render one side or portion non-porous. In this manner, the cells may be delivered to a specific porous portion of the scaffold for focused delivery to a wound site.

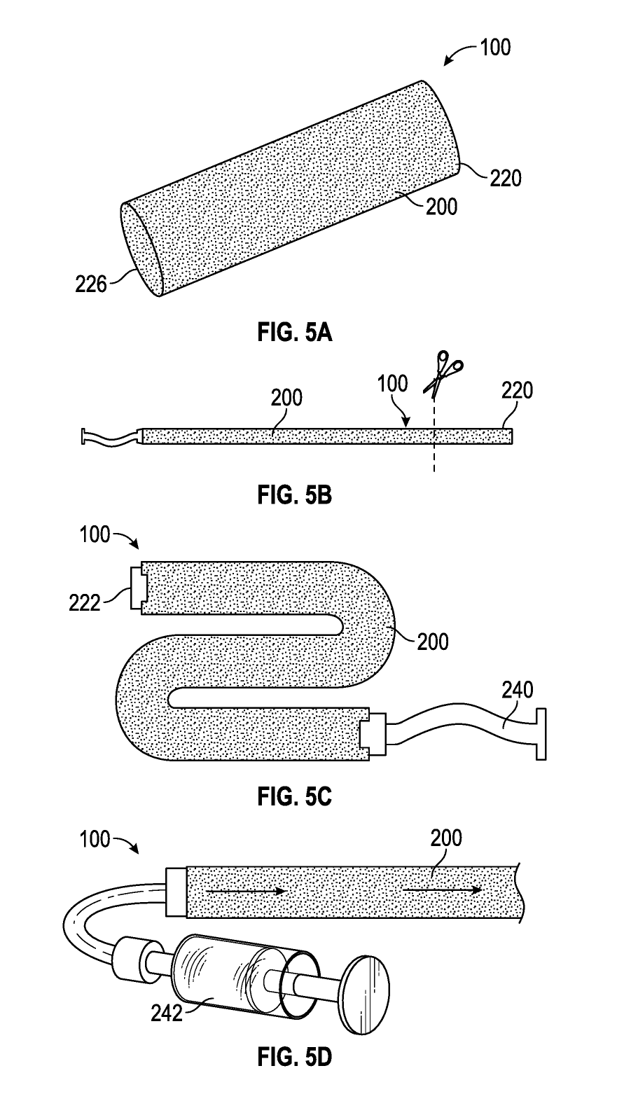

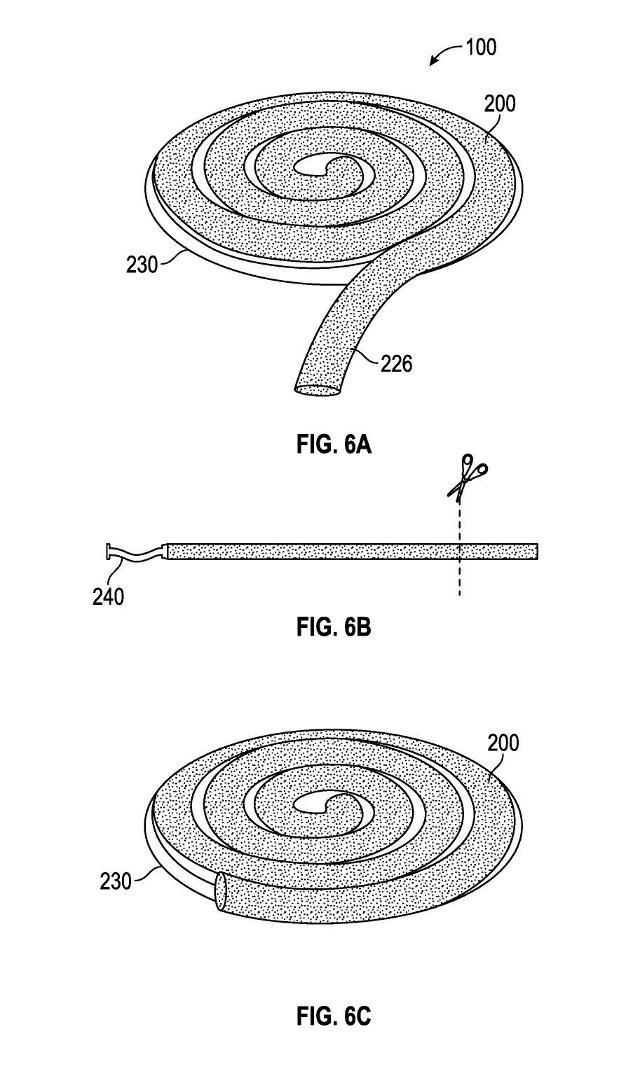

Referring generally to FIGS. 5A-8C, in other embodiments , the scaffold 100 can also be constructed in the form of a conduit or tube by methods including but not limited to those described above. In particular, the scaffold 100 includes a tubular body portion 200 that is flexible and porous. In certain embodiments, the tubular body portion 200 may be cross-linked, sutured, welded, or glued into a tubular configuration via ECM, polymer, plant-tissue, and the like.

In use, after measuring length of construct needed to treat wound, the tubular body portion 200 may be cut, closed or filtered at distal end 220, then autologous cells or allogeneic cells are added via a delivery means 242 to an opposite distal end 226, either outside wound or in situ. As construct is placed in a wound, a cover dressing, e.g., an island dressing or the like, or second piece of specified degradable material is placed on top to anchor tubular construct in place. In certain embodiments, the center of dressing may be fused, cross-linked or clipped shut.

More specifically, the tubular body portion 100 is cut to the desired length for the wound or injury site 230 and an injection port 240 is added to one end of the body 100 for cell administration. A filter 222 could be added to the other end of the tubular delivery system to permit passage of media or vehicle while preventing the release of cells. This filter could also be modified to offer resistance to flow in order to increase the internal hydrostatic pressure within the tube to force cell suspension into the potentially microporous walls of the tube. Once populated with cells, the tubular body portion 100 could be administered or implanted into the wound bed 230 or injury site.

In certain embodiments, the scaffold 10, 100 could also be constructed in situ; for example: the clinician adds the cell containing structure, such as the degradable tubular body 100 or the passage 44 of body portion 10, then adds a polymerizing degradable scaffold, e.g., fibrinogen and thrombin polymerizing into fibrin, to encase the wound 30.

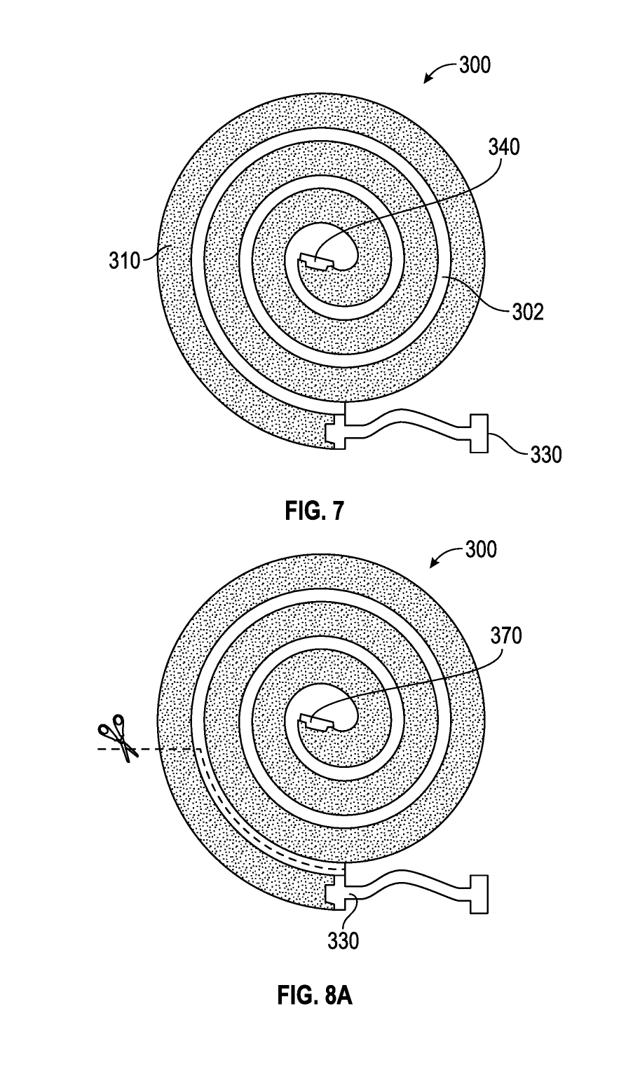

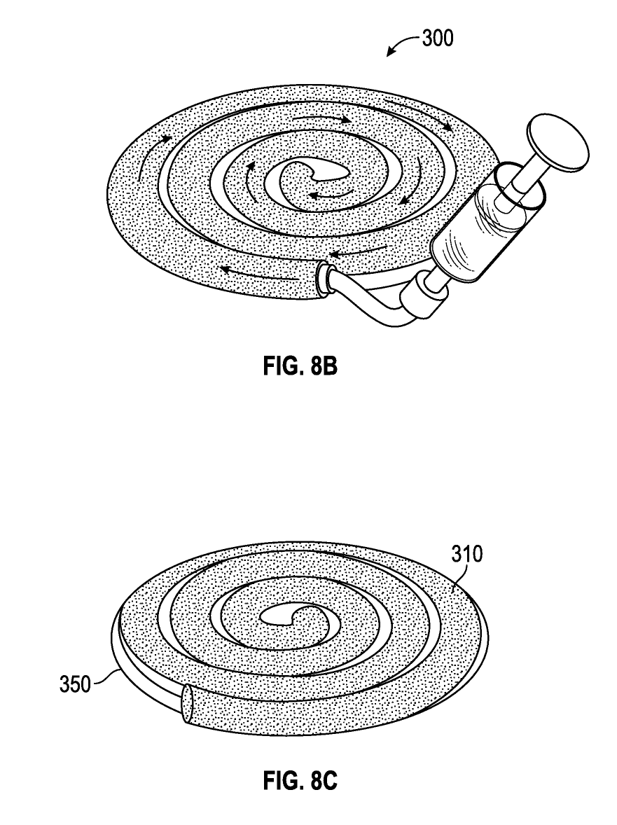

As shown in FIGS. 7 and 8A-8C, the scaffold 300 may have a tubular body portion 310 that is preformed, e.g., connected via material 302, into annular coil. The body 310 may contain and inlet, i.e., injection port, 330 and an outlet 340 with or without a filter 370. In use, the coil may be cut down to size prior to placement in a wound 350.

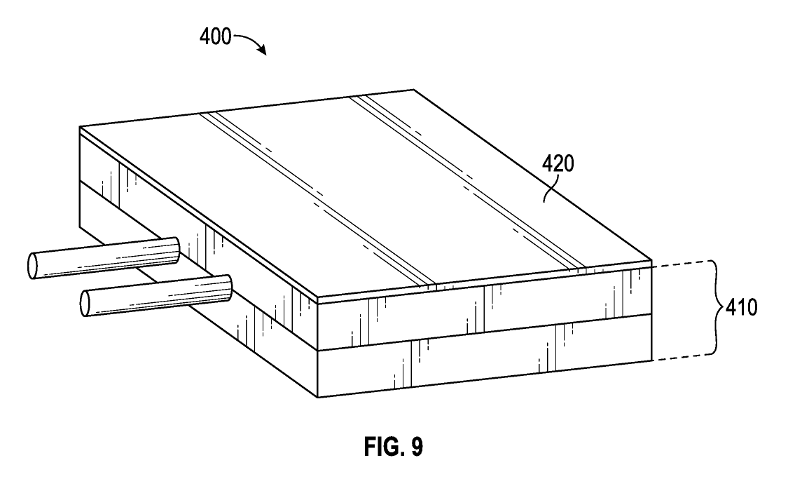

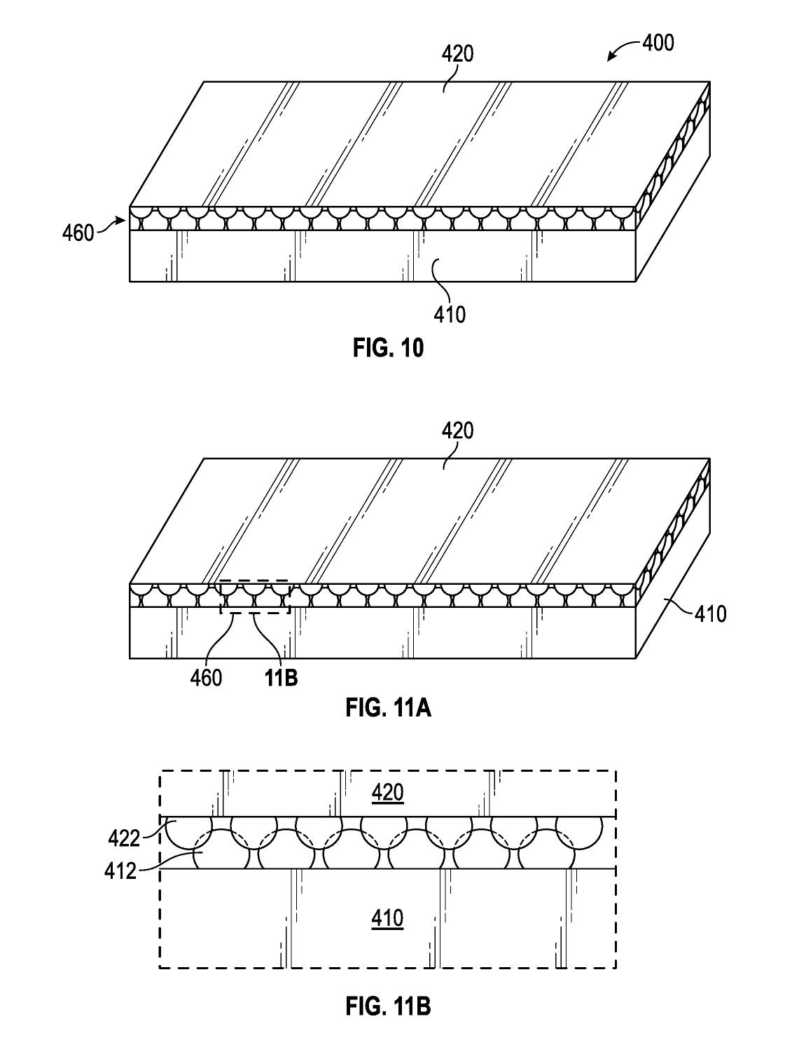

Referring now to FIGS. 9, 10 and 11A-11B, in embodiments, the body portion 410 may include a non-degradable silicone or polymer layer 420 that overlies the degradable bioactive scaffold 400 to prevent evaporative water loss while strengthening the scaffold for suturing or otherwise fixating the device to a patient. The polymer layer 420 may include one or more features that integrate into the underlying degradable scaffold, such that the silicone layer is released once the degradable scaffold has been sufficiently degraded to clinically warrant removal.

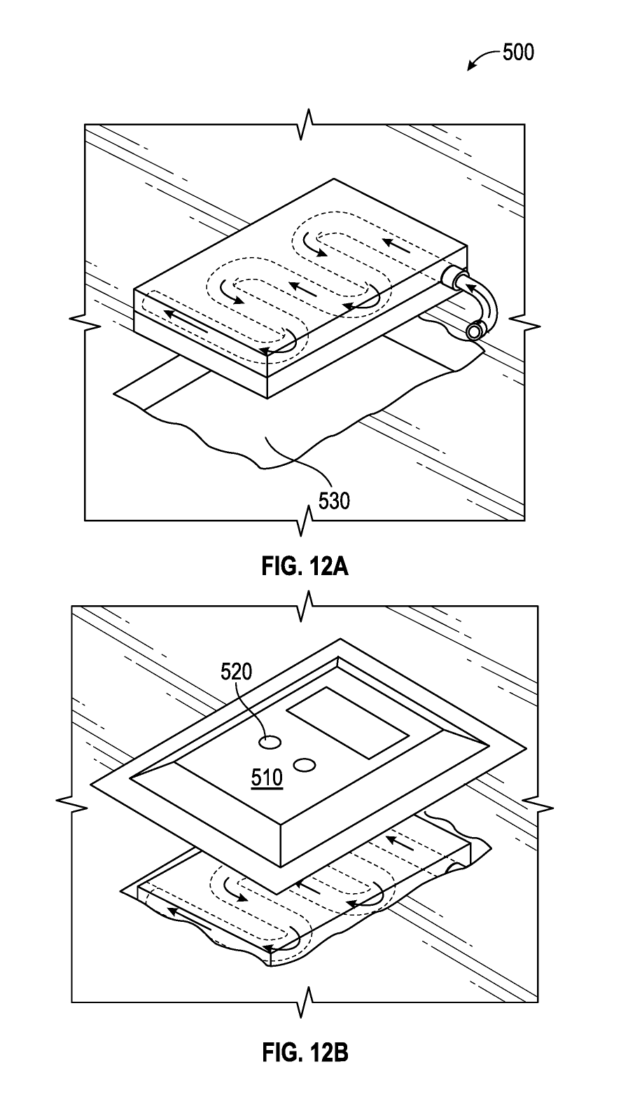

In particular, degradable structures 460 such as linkages, hook and loops or other features may be present that integrate the non-degradable layer into the underlying bioactive degradable body portion such that the non-degradable layer 420 is released once the degradable body portion 410 has been degraded and replaced by a healthy granulation tissue bed. As shown in FIGS. 11A and 11B, the structures 460 can be intercalated or interlocking degradable protrusions or loops 412 that fixedly mate or engage with a corresponding non-degradable protrusions or loops 422. As the degradable structure break down or lose mechanical integrity, they release the non-degradable structures and the attached non-degradable layer 420 from the degradable bioactive body portion 410.

In embodiments, once this has occurred the administration of an additional population of the same or different cells make take place. That is, embodiments contemplate a staged differential cell delivery, which is particularly useful for the treatment of injuries that involve the full thickness of the skin, which includes more than one cell population.

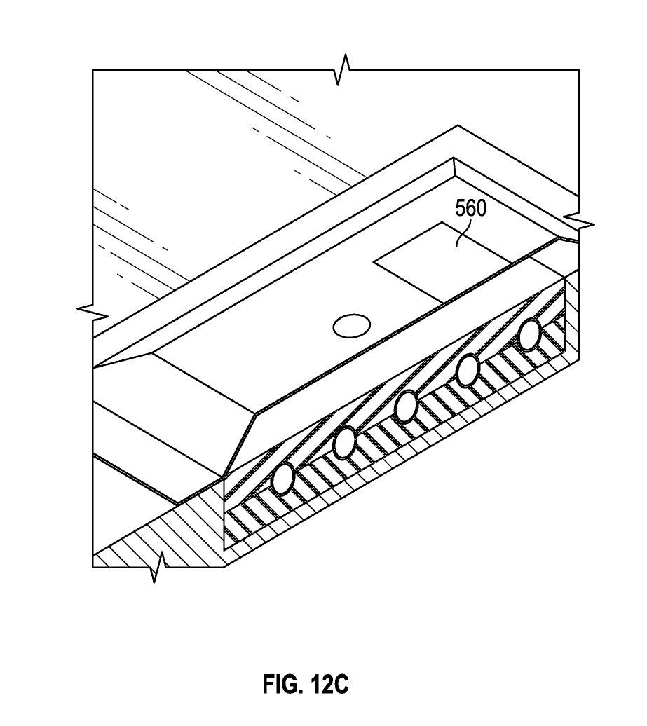

As shown in FIGS. 12A-12C, in certain embodiments, the bioactive scaffold 500 can be include a separate “incubator” layer or cover dressing 510 that can, for example, sense and/or control heat, humidity, pH, periwound atmosphere, or other vulnerary properties of the wound microenvironment to maintain or affect cell survival. This layer or dressing 510 would be placed over the bioactive scaffold 500 in the wound 530 and could provide persistent benefit as the scaffold degrades. Here, the degraded or degrading scaffold can provide signals, e.g., cytokines, attachment, and the incubator dressing will improve or alter the physiology of the cells it contains, thorough sensing and modulation of various parameters.

In embodiments, a sensor or suite of sensors 520 could be integrated to monitor scaffold degradation, wound healing or to actively aid in wound healing through modulation of parameters. These sensors could, for example, include 1) humidity, 2) gas detection 3) exudate monitoring (pH, hemoglobin, protein content and identity, bacterial contamination and microbial identity, 4) endogenous or exogenous (bacterial) proteolytic and or other enzyme identity and concentration or enzymatic activity, 6) pH, 7) temperature, 8) hydration, and 9) video (live, still, time-lapse) to assess scaffold breakdown, perfusion, granulation tissue formation, angiogenesis and/or wound reepithelialization, as well as other biomarkers for healing or impairment of healing.

In specific aspects, an incubator dressing can include a contact layer with one or more of a reservoir 560 for maintenance of hydration, relative humidity (RH), pH, elements for heating or cooling, sensors for O₂, CO₂, CH₃, and other biomarkers of infection, a pH sensor, a mechanical stimulator of connective tissue formation or ECM secretion. O₂, CO₂, and N₂ delivery systems may also be employed to select for or against aerobic or anaerobic species of pathogenic or commensal bacteria or microbes. Various aspects of incubator dressings suitable for use with embodiments of the inventive scaffold are more fully described in PCT US16/42788, the entirety of which is hereby incorporated by reference.

In certain embodiments, the porosity of the scaffold, e.g., the body portion, may be varied or altered to allow the passage of nutritive fluids and exchange of gases to support and/or foster the survival of added cells. Alteration of porosity could be performed to 1) affect the rate of degradation (by increasing the surface are to volume ratio), 2) provide a path for cell entry and population, or 3) allow passage of fluids such as exudate or exchange of gases O₂/CO₂ to foster the survival or growth of added or perfused cells. While embodiments increase the porosity for better mass transfer, the native architecture at the cellular level will not be affected. This improved structure will be beneficial in the fostering of survival and performance of added cells.

In particular embodiments, such as embodiments with a body portion that includes two layers, each layer may have a different porosity such that a side of the scaffold may be selected that has a porosity particularly suited for a specific wound etiology. In embodiments featuring a tubular body portion, the selection of surface orientation to place a luminal surface of the ECM biomaterial on the interior or exterior of the tube could be performed. More specifically, decellularized ECM biomaterials derived from the gastrointestinal tract and circulatory system may be utilized. These materials have directional permeability with the luminal surface showing tightly woven ECM structure in comparison to the more open weave ECM of the abluminal surface. By placing the luminal surface on the interior of the cross-linked tubular body portion, the migration of cells into the walls of the tubular body portion could be limited and the cells held for a longer time in the interior of the chambers created by the construct.

Alternatively, the more open structure of the abluminal surface could be placed on the internal surface (lumen) of the tubular body portion in order to permit migration of the added cells into the walls of the tube. This could be aided by addition of pressure against a filter as described above and as described for another application, specifically in the seeding of synthetic coronary bypass grafts.

Embodiments of the bioactive scaffold may also be utilized as a soft tissue graft that would not require a typically sized partial donor site from elsewhere on the patient. Such embodiments would include a secondary (or primary) port and path through the scaffold, e.g., through the body portion, to permit passage of circulating blood to provide nutrition, metabolic waste removal and gas exchange for the added cell population. If the cells were obtained through autologous harvest, the donor site would be lessened in comparison to the size typically required for a partial or split thickness skin graft (STSG) or other soft tissue graft. In trauma reconstructive and plastic surgery, this is a commonly performed procedure called a “flow through graft”.

Other embodiments permit the selection of specific cell types to address specific etiology underlying the wound pathology, providing patient-specific or wound-specific approach to wound care. In certain embodiments, the selection of specific cell types, e.g., optimal cells, based on the underlying pathophysiology is envisioned. In specific aspects, cell types may be selected based on the etiology of chronic wounds, e.g., neuropathic or diabetic foot ulcers, venous leg ulcers, decubitus or pressure ulcers, or acute trauma injuries or burns.

Moreover, single cell types or multiple cell cocktails could be added to the delivery system as needed by the clinician user. A clinician could select a keratinocyte cell source for superficial VLU, minor traumatic injury or first to second degree burns or fibroblasts for a deeper DFU, avulsive injury, or excised full thickness third degree burn. Depending on the status of the full thickness wound a second population of keratinocytes could be added to speed epithelization. Vascular endothelial cells could be added to promote angiogenesis in both examples above. Neuronal cell populations could be added to promote reinnervation. Immune cells from the circulation such as monocytes/macrophages and/or neutrophils could be added to speed proteolytic degradation of the construct or conditioned media or endotoxin exposure could be used to drive monocytes towards a regenerative phenotype over an inflammatory phenotype. For an internal implant, organ specific cell such as pancreatic islets or liver hepatocytes could be added prior to implantation. For soft tissue coverage of exposed orthopedic injuries, allogeneic or autologous muscle and or vascular endothelial or progenitor pericytes cells could be added to the cell construct.

In another embodiment, therapeutic cytokines to modulate healing, including but not limited to interleukins, may be provided. In addition, growth factors may be employed, these factors include platelet derived growth factor (PDGF), fibroblast growth factor (FGF), epidermal growth factor (EGF), connective tissue growth factor (CIGF), hepatocyte growth factor (HGF), insulin-like growth factor (IGF), stromal cell derived factor-1 (SDF-1), bone morphogenic proteins (BMPs), nerve growth factor (NGF) transforming growth factors (a,b), keratinocyte growth factor (KGF) or vascular endothelial growth factor (VEGF) in isolation or combination could be added to the cell delivery system to augment the survival, proliferation, engraftment or performance of the selected cell populations. It is of note that these labile signals would be directed at the added cell population and could generate secondary signal release from the cell population to augment or improve function. In addition, if stabilized, these incorporated signals could further enrich the breakdown products of the bioactive scaffold in the wound bed and enhance the migration, proliferation and vulnerary function of endogenous cells within the wound or injury site.

In another embodiments, bacteriostatic agents, antibacterials, antimicrobials, antibiotics or antifungals could be incorporated into the cell delivery system to prevent critical colonization with pathogenic or saprophytic microbes. Despite standard of care treatment, topical dermal wounds are generally considered non-sterile and colonized with low to high numbers of commensal and pathogenic bacteria and yeast. Addition of these therapeutic agents could prevent infection and premature breakdown of the scaffold, increased inflammation in response to infection, or risk of transmission to the treated tissue. Examples of antimicrobials include but are not limited to silver, copper, zinc, titanium oxide, chlorhexidine gluconate, polyhexamethylene biguanide, povidone iodine, cadexomer iodine, citric acid, hypochlorous acid, antimicrobial peptides, honey, glucose oxidase generated hydrogen peroxide, or hydrogen peroxide generated or held by other methods. Antimicrobial agents with selectivity for bacterial physiologic targets over eukaryotic cytotoxicity would be preferred.

In another embodiment, anti-inflammatory or pro-inflammatory signals or anti-scarring agents could be incorporated, to modulate endogenous inflammatory reaction, which is known in the field to involve the release of proteolytic enzymes that would speed the degradation of an ECM scaffold based cell delivery system. Examples could include but are not limited to steroids, steroidal anti-inflammatory drugs, inhibitors of cyclooxygenase (COX) 1 & 2, non-steroidal antiflammatory drugs (NSAIDs) including ibuprofen and naproxen sodium, and anti-oxidants such as ascorbic acid or carotenoids.

In another embodiment, macroporosity or microporosity could be increased or decreased by a clinician to speed or slow scaffold degradation, respectively. In a decellularized rather than reconstituted (solubilization followed by lyophilization) form, the native ECM typically shows differential porosity as described above. While this porosity range is evolutionarily optimized for the cell population of specific anatomical tissue cell types in the source tissue, it is unknown if this porosity range is optimal for the introduced selected cell populations as described for this scaffold based cell delivery system.

Therefore, the alteration of porosity using for example but not limited to proteolytic degradation, mechanical perforation, or freeze thaw fracture to increase the porosity of the ECM scaffold or cross-linking (as described in detail above), lamination (luminal surface to luminal surface, abluminal surface to abluminal surface, or luminal surface to abluminal surface)SCAFFOLD-, impregnation with synthetic polymers or ECM from another anatomical source, or collapsing matrix through serial freeze drying to decrease the porosity of the ECM scaffold.

In yet another embodiment, hemostatic agents could be added to limit bleeding upon implantation. It should be noted that the fibrin clot that would be expected to form is itself a provisional matrix, which could aid in engraftment of the cell delivery scaffold. Hemostatic agents could include but are not limited to chitosan, kaolin, denatured collagen (gelatin), fibrin glue, zeolite granules, or ECM altered and configured in a manner to make it hemostatic.

While the dimensions and types of materials described herein are intended to define the parameters of the invention, they are by no means limiting and are exemplary embodiments. Many other embodiments will be apparent to those of skill in the art upon reviewing the above description. The scope of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, terms such as “first,” “second,” “third,” “upper,” “lower,” “bottom,” “top,” etc. are used merely as labels, and are not intended to impose numerical or positional requirements on their objects.

Further, the limitations of the following claims are not written in means-plus-function format unless and until such claim limitations expressly use the phrase “means for” followed by a statement of function void of further structure. This written description uses examples to disclose several embodiments of the invention, including the best mode, and also to enable one of ordinary skill in the art to practice the embodiments of invention, including making and using any devices or systems and performing any incorporated methods. The patentable scope of the invention is defined by the claims, and may include other examples that occur to one of ordinary skill in the art. Such other examples are intended to be within the scope of the claims if they have structural elements that do not differ from the literal language of the claims, or if they include equivalent structural elements with insubstantial differences from the literal languages of the claims.

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional such elements not having that property.

Since certain changes may be made in the above-described invention, without departing from the spirit and scope of the invention herein involved, it is intended that all of the subject matter of the above description shown in the accompanying drawings shall be interpreted merely as examples illustrating the inventive concept herein and shall not be construed as limiting the invention.

What is claimed is:

1. A bioactive scaffold for cell delivery to a wound comprising:
a degradable body portion configured to be placed within a wound bed, the body portion including a least one extracellular matrix biomaterial.

2. The bioactive scaffold of claim 1 wherein the degradable body portion further includes at least one cytokine, drug, or antimicrobial agent.

3. The bioactive scaffold of claim 1 wherein the degradable body portion further comprises:
an inlet configured to receive cells;
a passage operatively connected to the inlet, the passage located within the degradable body portion; and
wherein the passage is configured to provide cells to the body portion for delivery to the wound.

4. The bioactive scaffold of claim 3, further comprising:
an outlet operatively connected to the passage, the outlet allowing for the removal of cell delivery media from the body portion.

5. The bioactive scaffold of claim 4 wherein the passage further includes a filter configured to permit exit of cell delivery media from the outlet while preventing the release of cells.

6. The bioactive scaffold of claim 5 wherein the filter is selectively adjustable to provide flow resistance in order to increase internal hydrostatic pressure within the passage to regulate cell delivery.

7. The bioactive scaffold of claim 3 wherein the passage is formed from an extracellular matrix biomaterial.

8. The bioactive scaffold of claim 3 wherein the passage is formed from the extracellular matrix biomaterial of the degradable body portion.

9. The bioactive scaffold of claim 7 wherein the passage and body portion extracellular matrix biomaterials are different from one another.

10. The bioactive scaffold of claim 1 further comprising:
a cover portion including an incorporated border to secure the scaffold to a wound bed.

11. The bioactive scaffold of claim 1 wherein the body portion includes a first layer and a second layer.

12. The bioactive scaffold of claim 11 wherein the first and second layers are glued, ultrasonically welded, laser welded, thermally melted or cross-linked together.

13. The bioactive scaffold of claim 11 wherein the first and second layers are comprised of different extracellular matrix biomaterials.

14. The bioactive scaffold of claim 1 wherein the scaffold is configured to be grafted in place.

15. The bioactive scaffold of claim 14 wherein the body portion further comprises:
a passage for the circulation of blood to and/or from the wound.

16. The bioactive scaffold of claim 1 wherein the body portion has first and second opposing sides, the opposing sides having different porosities.

17. The bioactive scaffold of claim 11 wherein the first layer is degradable and the second layer is non-degradable; and
the first and second layers are secured together by at least one degradable structure; and
wherein the degradable structure is configured to degrade when the bioactive scaffold is in use to release the first layer from the second layer.

18. The bioactive scaffold of claim 1 further comprising:
a cover dressing configured to sense and/or control heat, humidity, pH, periwound atmosphere, or other vulnerary properties of the wound microenvironment to maintain or affect cell survival.

19. The bioactive scaffold of claim 18 wherein the cover dressing includes a reservoir for altering wound environment; and
at least one sensor, the sensor being configured to monitor at least one of hydration, humidity, gas detection, exudate monitoring including pH, hemoglobin, protein content and identity, bacterial contamination and microbial identity, endogenous or exogenous proteolytic and or other enzyme identity, and concentration or enzymatic activity, temperature, scaffold breakdown, perfusion, granulation tissue formation, angiogenesis and/or wound reepithelialization.

20. The bioactive scaffold of claim 1 further comprising:
a non-degradable cover layer that overlies the body portion to prevent evaporative water loss while strengthening the scaffold.

21. A bioactive scaffold for cell delivery to a wound comprising:
a degradable, flexible and porous tubular body portion configured to be placed within a wound bed, the body portion including an extracellular matrix biomaterial; and
wherein the tubular body portion has a substantially hollow interior.

22. The bioactive scaffold of claim 21 wherein the tubular body portion includes at least one cytokine, drug, or antimicrobial agent.

23. The bioactive scaffold of claim 21 wherein the tubular body portion further comprises:
an inlet on a distal end of the tubular body portion, the inlet configured to receive cells.

24. The bioactive scaffold of claim 23, further comprising:
an outlet on an opposite distal end of the tubular body portion, the outlet allowing for the removal of any cell delivery media.

25. The bioactive scaffold of claim 24 wherein the tubular body portion further includes a filter configured to permit exit of cell delivery media from the outlet while preventing the release of cells.

26. The bioactive scaffold of claim 25 wherein the filter is selectively adjustable to provide flow resistance in order to increase internal hydrostatic pressure within the tubular body portion to regulate cell delivery.

27. The bioactive scaffold of claim 21 further comprising:
a cover portion including an adhesive border to secure the scaffold to a wound bed.

28. The bioactive scaffold of claim 21 wherein tubular body portion is coiled such that the scaffold is substantially annular in shape.

29. The bioactive scaffold of claim 21 wherein the scaffold is configured to be grafted in place.

30. A method of delivering cells to a wound to facilitate healing, the method comprising:
placing a bioactive scaffold for cell delivery within a wound bed, the scaffold having a degradable body portion that includes at least one extracellular matrix biomaterial; and
delivering cells to the wound via the body portion of the scaffold.

31. The method of claim 30 further comprising:
selecting cells to be delivered based on an underlying wound pathology.

32. The method of claim 30 further comprising:
selecting a bioactive scaffold having a specific porosity based on an underlying wound pathology.

33. The method of claim 30 further comprising:
adjusting flow resistance within the body portion in order to increase internal hydrostatic pressure to regulate cell delivery.

34. The method of claim 30 further comprising:
adjusting the size of dressing based on the size of the wound bed.
